(19) United States
(12) Patent Application Publication
(10) Pub. No.: US 2005/0240009 A1
(43) Pub. Date: Oct. 27, 2005

(54) T-cell epitopes in staphylococcal enterotoxin b

(76) Inventors: Francis J. Carr, Balmedie (GB); Matthew Baker, Littleport, Ely (GB); Graham Carter, Newmachar (GB)

Correspondence Address: OLSON & HIERL, LTD., 20 NORTH WACKER DRIVE, 36TH FLOOR, CHICAGO, IL, 60606, US

(21) Appl. No.: 10/525,113

(22) PCT Filed: Aug. 18, 2003
(86) PCT No.: PCT/EP03/09116

§ 371 Date: Feb. 18, 2005

(30) Foreign Application Priority Data
Aug. 21, 2002 (EP) 02018229.1

(51) Int. Cl. A61K 39/02; C07K 14/31; A61K 39/085

(52) U.S. Cl. 530/350; 424/190.1; 424/237.1

(57) ABSTRACT

The present invention relates to the field of immunology. The invention identifies determinants on staphylococcal enterotoxin B (SEB) able to evoke an immune response. In particular the invention is concerned with the identification of epitopes for T-cells in SEB. The invention relates furthermore to T-cell epitope peptides derived from SEB by means of which it is possible to create modified SEB variants with reduced immunogenicity.

### FIELD OF THE INVENTION

The present invention relates to the field of immunology. The invention identifies determinants on staphylococcal enterotoxin B (SEB) able to evoke an immune response. In particular the invention is concerned with the identification of epitopes for T-cells in SEB. The invention relates furthermore to T-cell epitope peptides derived from SEB by means of which it is possible to create modified SEB variants with reduced immunogenicity.

### BACKGROUND OF THEE INVENTION

There are many instances whereby the efficacy of a therapeutic protein is limited by an unwanted immune reaction to the therapeutic protein. Several mouse monoclonal antibodies have shown promise as therapies in a number of human disease settings but in certain cases have failed due to the induction of significant degrees of a human anti-murine antibody (HAMA) response [Schroff, R. W. et al (1985) Cancer Res. 45: 879-885; Shawler, D. L. et al (1985) J. Immunol. 135: 1530-1535]. For monoclonal antibodies, a number of techniques have been developed in attempt to reduce the HAMA response [WO 89/09622; EP 0239400; EP 0438310; WO 91/06667]. These recombinant DNA approaches have generally reduced the mouse genetic information in the final antibody construct whilst increasing the human genetic information in the final construct. Notwithstanding, the resultant “humanised” antibodies have, in several cases, still elicited an immune response in patients [Issacs J. D. (1990) Sem. Immunol. 2: 449,456; Rebello, P. R. et al (1999) Transplantation 68: 1417-1420].

Antibodies are not the only class of polypeptide molecule administered as a therapeutic agent against which an immune response may be mounted. Even proteins of human origin and with the same amino acid sequences as occur within humans can still induce an immune response in humans. Notable examples amongst others include the therapeutic use of granulocyte-macrophage colony stimulating factor [Wadhwa, M. et al (1999) Clin. Cancer Res. 5: 1353-1361] and interferon alpha 2 [Russo, D. et al (1996) Bri. J. Haem. 94: 300-305; Stein, R. et al (1988) New Engl. J. Med. 318: 1409-1413]. In such situations where these human proteins are immunogenic, there is a presumed breakage of immunological tolerance that would otherwise have been operating in these subjects to these proteins.

A sustained antibody response to a therapeutic protein requires the stimulation of T-helper cell proliferation and activation. T-cell stimulation requires the establishment of a T-cell synapse between a T-cell and an antigen presenting cell (APC). At the core of the synapse is the T-cell receptor (TCR) on the T-cell engaged with a peptide MHC class II complex on the surface of the APC. The peptide is derived from the intracellular processing of the antigenic protein. Peptide sequences from protein antigens that can stimulate the activity of T-cells via presentation on MHC class II molecules are the termed “T-cell epitopes”. Such T-cell epitopes are commonly defined as any amino acid residue sequence with the ability to bind to MHC Class II molecules. Implicitly, a “T-cell epitope” means an epitope which when bound to MHC molecules can be recognised by a TCR, and which can, at least in principle, cause the activation of these T-cells by engaging a TCR to promote a T-cell response. It is understood that for many proteins a small number of T-helper cell epitopes can drive T-helper signalling to result in sustained, high affinity, class-switched antibody responses to what may be a very large repertoire of exposed surface determinants on the therapeutic protein.

T-cell epitope identification is recognised as the first step to epitope elimination, and it is highly desired to identify T-cell epitopes in therapeutic proteins. Patent applications WO98/52976 and WO00/34317 teach computational threading approaches to identifying polypeptide sequences with the potential to bind a sub-set of human MHC class II DR allotypes. In these teachings, predicted T-cell epitopes are removed by the use of judicious amino acid substitution within the protein of interest. However with this scheme and other computationally based procedures for epitope identification [Godkin, A. J. et al (1998) J. Immunol. 161: 850-858; Sturniolo, T. et al (1999) Nat. Biotechnol. 17: 555-561], peptides predicted to be able to bind MHC class II molecules may not function as T-cell epitopes in all situations, particularly, in vivo due to the processing pathways or other phenomena. In addition, the computational approaches to T-cell epitope prediction have in general not been capable of predicting epitopes with DP or DQ restriction.

Equally, in vitro methods for measuring the ability of synthetic peptides to bind MHC class II molecules, for example using B-cell lines of defined MHC allotype as a source of MHC class II binding surface [Marshall K. W. et al. (1994) J. Immunol. 152:4946-4956; O'Sullivan et al (1990) J. Immunol. 145: 1799-1808; Robadey C. et al (1997) J. Immunol 159: 3238-3246], may be applied to MHC class II ligand identification. However, such techniques are not adapted for the screening multiple potential epitopes to a wide diversity of MHC allotypes, nor can they confirm the ability of a binding peptide to function as a T-cell epitope.

Recently techniques exploiting soluble complexes of recombinant MEC molecules in combination with synthetic peptides have come into use [Kern, F. et al (1998) Nature Medicine 4:975-978; Kwok, W. W. et al (2001) TRENDS in Immunol. 22:583-588]. These reagents and procedures are used to identify the presence of T-cell clones from peripheral blood samples from human or experimental animal subjects that are able to bind particular MHC-peptide complexes and are not adapted for the screening multiple potential epitopes to a wide diversity of MHC allotypes.

Biological assays of T-cell activation remain the best practical option to providing a reading of the ability of a test peptide/protein sequence to evoke an immune response. Examples of this kind of approach include the work of Petra et al using T-cell proliferation assays to the bacterial protein staphylokinase, followed by epitope mapping using synthetic peptides to stimulate T-cell lines [Petra, A. M. et al (2002) J. Immunol. 168: 155-161]. Similarly, T-cell proliferation assays using synthetic peptides of the tetanus toxin protein have resulted in definition of immunodominant epitope regions of the toxin [Reece J. C. et al (1993) J. Immunol. 151: 6175-6184]. W099/53038 discloses an approach whereby T-cell epitopes in a test protein may be determined using isolated sub-sets of human immune cells, promoting their differentiation in vitro and culture of the cells in the presence of synthetic peptides of interest and measurement of any induced proliferation in the cultured T-cells. The same technique is also described by Stickler et al [Stickler, M. M. et al (2000) J. Immunotherapy 23:654-660], where in both instances the method is applied to the detection of T-cell epitopes within bacterial subtilisin. Such a technique requires careful application of cell isolation techniques and cell culture with multiple cytokine supplements to obtain the desired immune cell sub-sets (dendritic cells, CD4+ and or CD8+ T-cells).

As depicted above and as consequence thereof, it would be desirable to identify and to remove or at least to reduce T-cell epitopes from a given in principal therapeutically valuable but originally immunogenic peptide, polypeptide or protein. One of these potential therapeutically valuable molecules is staphylococcal enterotoxin B (SEB).

SEB is a member of the family of enterotoxins produced by Staphylococcus aureus. Other members include serologically distinct proteins, designated A, C₁, C₂, C₃, D, E and F. These proteins are recognised as the causative agents of staphylococcal food poisoning. One of the therapeutic interests in this class of protein stems from their ability to function as “superantigens” that is, molecules able to stimulate the activity of human T-cells. Their therapeutic potential has been tested in a number of clinical trials for cancer where the objective has been to achieve enhanced T-cell activation to result in immune mediated suppression of tumour cell growth. In some cases the toxin molecules have been linked to antibodies to provide cell specific targeting [Dohlstein, M et al (1994) PNAS USA 91: 8945-8949; Giantonio, B. J. et al (1997) J. Clin. Oncol. 15: 1994-2007; Hansson, J. et al (1997) PNAS USA 94: 2489-2494; Alpaugh, K. R. et al (1998) Clin. Cancer Res. 4: 1903-1914].

The present invention is concerned primarily with the enterotoxin B. The mature amino acid sequence of SEB contains 237 amino acid residues and depicted in single-letter code comprises the following sequence:

ESQPDPKPDELHKSSKFTGLMENMKVLYDDNHVSAINVKSIDQFLYFDLI
YSIKDTKLGNYDNVRVEFKNKDLADKYKDKYVDVFGANYYYQCYFSKKTN
DINSHQTDKRKTCMYGGVTEHNGNQLDKYRSITVRVFEDGKNLLSFDVQT
NKKKVTAQELDYLTRHYLVKNKKLYEFNNSPYETGYIKFIENENSFWYDM
MPAPGDKFDQSKYLMMYNDNKMVDSKDVKIEVYLTTKKK

As “superantigens” the staphylococcal enterotoxins are the most powerful T cell mitogens 30 known eliciting strong polygonal proliferation at concentrations 10³ lower than such conventional T cell mitogens as phytohemagglutinin. All stimulate a large proportion human CD4+ and CD8+ T cells. Their ability to stimulate T-cells is tightly restricted by the MHC class II antigens. It is understood that the staphylococcal enterotoxins, and the other superantigen toxins bind directly to the T cell receptor and to MHC class II. These two structures are brought into contact, thus stimulating T cell activation via the V_{□} region of the T cell receptor mimicking strong alloreactive response. Recognition of most conventional antigenic peptides bound to MHC proteins involves contributions from all the variable components of the T cell receptor. In contrast, the toxins stimulate T cells almost exclusively via the V_{□} region of the T cell receptor. The toxins may be thought of as clamps engaging the sides of the MHC class II and V_{□} to bring into close proximity the surfaces of the T cell receptor and MHC that would ordinarily contact each other during T cell/APC synapse formation.

These and other particular properties of the superantigen molecules have prompted their use in a number of different experimental therapeutic strategies, including cancer therapies. In the case of the SEB toxin, a series of US patents; U.S. Pat. No. 6,180,097; U.S. Pat. No. 5,728,388; U.S. Pat. No. 6,338,845; U.S. Pat. No. 6,221,351; U.S. Pat. No. 6,126,945 and equivalents WO93/24136; WO98/26747; EP1103268 and EP0511306 all due to Terman and colleagues, collectively describe in detail the art with regard to use of SEB genes, SEB proteins, including carboxymethylated SEB protein and SEB-antibody conjugates and fusion proteins. All are directed to methods and or compositions for the purpose of inducing cancer cell killing effects and cancer therapy.

Thus for example, EP0511306 claims use of enterotoxin molecules including SEB, homologues of SEB and SEB fragments having essentially the same biological activity as a superantigen and SEB conjugates with monoclonal antibodies.

Such molecules and conjugates are provided for use as cancer therapies and may be effective as such. However owing to the foreignness of the SEB (and also possibly any conjoined antibody component) to the human immune system there is considerable likelihood of an immune response being evoked which may not limit the effectiveness of any first administered dose, but may well limit the effectiveness or cause significant deleterious side effects on subsequent doses. The claimed agents are directed to cancer patients only. For many such patients their immune system may be suppressed as a consequence of previous therapeutic regimens or as a direct result of their disease, and therefore the immunogenic consequences of the SEB based therapy may be lessened. However, such a limitation may not exist in other patients where an SEB based therapy may be helpful. It is an objective of the present invention to define the immunogenic regions of the SEB molecule as a first step to providing SEB based compositions with a reduced potential to induce harmful immunogenic responses. Such compositions would be applicable to a wider variety of clinical indications, including non-cancer diseases, than is currently the case.

By contrast, U.S. Pat. No. 6,528,051 contemplates using SEB as an antigen against which a specific and protective immune response is mounted. The SEB is administered as a colloidal gold complex.

Similarly, U.S. patent application 20010046501A1 advances use of mixed SEA/SEB compositions in a therapeutic or prophylactic treatment regime for infectious disease indications. The approach provides compositions and treatment schedules able to enhance specific immune responses to antigens by depletion of naïve (non-activated) T-cell populations.

More recently, U.S. patent application 20030009015A1 provides superantigen vaccine preparations in which the superantigen attributes are absent but the structure sufficiently intact to be recognised by the immune system to effect a protective vaccination. SEB molecules containing substitutions within either the MHC class II binding region or the TCR binding region are described and considered sufficient to achieve the desired outcome. The substitutions contemplated, using single letter code, include 61A, 67Q, 89A, 94A and 115A.

In general, parallel strategies have been adopted exploiting the SEA toxin although for example, U.S. patent application 20030039655A1 contemplates SEA-antibody conjugates in which the SEA moiety contains amino acid substitutions at surface exposed residues with the effect of reducing sero-reactivity. In contrast to the present case, this application is concerned with surface determinant of the SEA molecule able to interact with host antibodies and is not directed to T-cell eptiopes in SEB.

From the foregoing it can be seen that where others have provided SEB molecules including modified SEB molecules, these teachings do not address the importance of T cell epitopes to the immunogenic properties of the protein nor have been conceived to directly influence said properties in a specific and controlled way according to the scheme of the present invention.

Accordingly, it is a particular objective of the present invention to provide modified SEB proteins in which the immune characteristic is modified by means of reduced numbers of potential T-cell epitopes. This immune characteristic is distinct from the functional capability of the whole protein molecule to act as an inducer of T-cell activity via MHC-TCR cross-linking. Rather it is an objective of the present invention to provide for SEB molecules with a retained superantigen activity but a reduced ability to induce a neutralising immune response to SEB administered therapeutically and especially a T-cell mediated neutralising antibody response.

The provenance or location of T-cell epitopes within a linear protein sequence is referred to herein as an “epitope map”. It is an objective of the present invention to provide an epitope map for SEB.

It is a further objective of the invention to provide SEB analogues in which the previously mapped T-cell epitopes are compromised in their ability to function as MHC class II ligands and or activate T-cells in combination with MHC class II molecules. It is highly desired to provide SEB with reduced or absent potential to induce an immune response in the human subject and it is therefore a particular objective of the present invention to provide modified SEB proteins in which the immune characteristic is modified by means of reduced numbers of potential T-cell epitopes.

In summary the invention relates to the following issues:
using a panel of synthetic peptides in a naïve T-cell assay to map the immunogenic region(s) of SEB;
construction of a T-cell epitope map of SEB protein using PBMC isolated from 20 or more healthy donors and a screening method involving the steps comprising:
   i) antigen stimulation iii vitro using synthetic peptide immunogens at two or more concentrations of peptide for a culture period of up to 7 days; using PBMC preparations containing physiologic ratios of T-cell to antigen presenting cells and ii) measurement of the induced proliferation index by any suitable method;
SEB derived peptide sequences able to evoke a stimulation index of greater than 1.8 and preferably greater than 2.0 in a naïve T-cell assay,
SEB derived peptide sequences having a stimulation index of greater than 1.8 and preferably greater than 2.0 in a naïve T-cell assay wherein the peptide is modified to a minimum extent and tested in the naïve T-cell assay and found to have a stimulation index of less than 2.0;
SEB derived peptide sequences sharing 100% amino acid identity with the wild-type protein sequence and able to evoke a stimulation index of 1.8 or greater and preferably greater than 2.0 in a T-cell assay;
an accordingly specified SEB peptide sequence modified to contain less than 100% amino acid identity with the wild-type protein sequence and evoking a stimulation index of less than 2.0 when tested in a T-cell assay;
a SEB molecule in which the immunogenic regions have been mapped using a T-cell assay and then modified such that upon re-testing in a T-cell assay the modified protein evokes a stimulation index smaller than the parental (non-modified) molecule and most preferably less than 2.0;
a modified molecule having the biological activity of SEB and being substantially non-immunogenic or less immunogenic than any non-modified molecule having the same biological activity when used in vivo;

an accordingly specified molecule wherein alteration is conducted at one or more residues from the string of contiguous residues defined herein as epitope region R1, R2 and R3:

R1:; KFTGLMENMKVLYDDNHVSAI
R2:; QFLYFDLIYSIKDTKLGNYDNVRV
R3: NKDLADKYKDKYVDVFGANYYYQCYFSKKTNDI

an accordingly specified molecule wherein alteration is conducted at one or more residues from the string of contiguous residues defined herein as preferred epitope region R1a, R1b, R1c and comprising the sequence:

R1a, KFTGLMENMKVLYDD
R1b:, GLMENMKVLYDDNHV
R1c:. ENMKVLYDDNHVSAI

an accordingly specified molecule wherein alteration is conducted at one or more residues from the string of contiguous residues defined herein as preferred epitope region R2a and comprising the sequence SIKDTKLGNYDNVRV
an accordingly specified molecule wherein alteration is conducted at one or more residues from the string of contiguous residues defined herein as preferred epitope region R3a and comprising the sequence DKYVDVFGANYYYQC
a peptide molecule comprising 13-15 consecutive residues from any of sequences R1a,b,c-R3a, or R1-R3:
a peptide molecule comprising 13-15 consecutive residues from any of sequences identified in Table 1 herein;

a modified SEB molecule comprising the amino acid sequence of Formula I:

X⁰ESQPDPKPDELHKSSKFTGL¹ENX²KVLX³DDNHVSAINVKSIDQFLY
FDLIYSX⁴KDTKX⁵GNYDNVRVEFKNKDLADKYKDKX⁶X⁷DX⁸X⁹GANYY
YQCYFSKKTNDINSHQTDKRKTCMYGGVTEHNGNQLDKYRSITVRVFEDG
KNLLSFDVQTNKKKVTAQELDYLTRHYLVKNKKLYEFNNSPYETGYIKFI
ENENSFWYDMMPAPGDKFDQSKYLMMYNDNKMVDSKDVKIEVYLTTKKK,
wherein

X⁰ is hydrogen or a targeting moiety such as an antibody, an antibody domain [Fab′, F(ab)2′, scFv, Fc-domain], or another protein or polypeptide;
X¹=A, G, P or M;
X²=A, G, P, or M;
X³=T, A, D, E, G, H, K N, P, Q, R, S, or Y;
X⁴=A, or I;
X⁵═H, or L;
X⁶=T, A, D, E, G, H, K N, P, Q, R, S, or Y;
X⁷═H, or V;
X⁸=A, P, G, or V;
X⁹=T, H, or F;
whereby simultaneously X¹=M, X²=M, X³═Y, X⁴═Y, X⁵=L, X⁶═Y, X⁷═V, X⁸═V and X⁹═F are excluded.
a peptide molecule of above sharing greater than 80% amino acid identity with any of the peptide sequences derived from epitope regions R1-R3, or R1a-R3a;
a peptide molecule of above sharing greater than 80% amino acid identity with any of the peptide sequences derived from the peptide sequences identified in Table 1 herein;
peptide sequences as above able to bind MHC class II;
a pharmaceutical composition comprising any of the peptides or modified peptides of above having the activity of binding to MHC class II
a DNA sequence or molecule which codes for any of said specified modified molecules as defined above and below;
a pharmaceutical composition comprising a modified molecule having the biological activity of SEB;
a pharmaceutical composition as defined above and/or in the claims, optionally together with a pharmaceutically acceptable carrier, diluent or excipient;
a method for manufacturing a modified molecule having the biological activity of SEB comprising the following steps: (i) determining the amino acid sequence of the polypeptide or part thereof; (ii) identifying one or more potential T-cell epitopes within the amino acid sequence of the protein by any method including determination of the binding of the peptides to MHC molecules using in vitro or in silico techniques or biological assays; (iii) designing new sequence variants with one or more amino acids within the identified potential T-cell epitopes modified in such a way to substantially reduce or eliminate the activity of the T-cell epitope as determined by the binding of the peptides to MHC molecules using in vitro or in silico techniques or biological assays; (iv) constructing such sequence variants by recombinant DNA techniques and testing said variants in order to identify one or more variants with desirable properties; and (v) optionally repeating steps (ii)-(iv);
an accordingly specified method, wherein step (iii) is carried out by substitution, addition or deletion of 1-9 amino acid residues in any of the originally present T-cell epitopes;
an accordingly specified method, wherein the alteration is made with reference to an homologous protein sequence and/or in silico modelling techniques;
a peptide sequence consisting of at least 9 consecutive amino acid residues of a T-cell epitope peptide as specified above and its use for the manufacture of SEB having substantially no or less immunogenicity than any non-modified molecule and having the biological activity of SEB when used in vivo;
a concerted method for mapping the location of T-cell epitopes in SEB using naïve T-cell activation assays and a computational scheme simulating the binding of the peptide ligand with one or more MHC allotypes;
a method for locating T-cell epitopes in SEB comprising the following steps;
   i) use of naïve T-cell activation assays and synthetic peptides collectively encompassing the protein sequence of interest to identify epitope regions capable of activating T-cells;
   ii) use of a computational scheme simulating the binding of the peptide ligand with one or more MHC allotypes to analyse the epitope regions identified in step (i) and thereby identify MHC class II ligands within the epitope region;
   iii) use of a computational scheme simulating the binding of the peptide ligand with one or more MHC allotypes to identify sequence analogues of the MHC ligands encompassed within the epitope region(s) which no longer bind MHC class II or bind with lowered affinity to a lesser number of MHC allotypes;
   iv) use of naïve T-cell activation assays and synthetic peptides encompassing entirely or in collection encompassing the epitope regions identified within the protein of interest and testing the sequence analogues in naïve T-cell activation assay in parallel with the wild-type (parental) sequences;
a method according to the above scheme wherein steps (ii) and (iii) are carried out using a computational approach as taught by WO 02/069232;
a method according to the above scheme whereby step (iv) is optionally conducted;
a method according to the above scheme where the naïve T-cell activation assay is conducted using PBMC cells derived from around 20 or more unrelated donors;
a method according to the above scheme where the location of a T-cell epitope is found when a stimulation index score of around 2.0 is observed in two or more independent donor samples;
a method according to the above scheme where the location of a T-cell epitope is found when a stimulation index score of around 2.0 is observed in two or more independent donor samples;
a method according to the above scheme where the location of a T-cell epitope is found when a stimulation index score of around 2.0 is observed in two or more independent donor samples and where one or more MHC class II ligands can be identified within the same sequence locale using a computational system;
a method according to the above scheme whereby the computational system is according to the method as taught by WO 02/069232;

### DETAILED DESCRIPTION OF THE INVENTION

According to the first embodiment of the invention there is provided a T-cell epitope map of SEB. The epitope map of SEB has utility in enabling the design of SEB analogues in which amino acid substitutions have been conducted at specific positions and with specific residues to result in a substantial reduction in activity or elimination of one or more potential T-cell epitopes from the protein. The present invention provides examples of suitable substitutions within the most immunogenic regions of the parent molecule and such substitutions are considered embodiments of the invention.

Co-owned application WO 02/069232 used an in silico technique to define MHC class II ligands for multiple proteins of therapeutic interest. However, for reasons such as the requirement for proteolytic processing and other physiologic steps leading to the presentation of immunogenic peptides in vivo, it is clear that a relatively minor sub-set of the entire repertoire of peptides definable by computer-based schemes will have ultimate biological relevance. The inventors have established that ex vivo human T-cell activation assays may be used to identify the regions within the protein sequence of SEB that are able to support T-cell activation and are thereby most biologically relevant to the problem of immunogenicty in this protein. The epitope map of SEB disclosed herein has been derived by application of such an approach and the method as disclosed is accordingly also an embodiment of the present invention.

According to the method, synthetic peptides are tested for their ability to evoke a proliferative response in human T-cells cultured in vitro. The T-cells are present within peripheral blood mononuclear cell (PBMC) layer readily obtainable by well known means from whole blood samples. Moreover the PBMC preparation contains physiological ratios of T-cells and antigen presenting cells and is therefore a good source of materials with which to conduct a surrogate immune reaction in vitro. The inventors have established that in the operation of such an assay, a stimulation index closly approaching or exceeding 2.0 is a useful measure of induced proliferation. The stimulation index (SI) is conventionally derived by division of the proliferation score (e.g. counts per minute of radioactivity if using for example ³H-thymidine incorporation) measured to the test peptide by the score measured in cells not contacted with a test peptide. Peptides which evoke no response give SI=1.0 although in practice SI values in the range 0.8-1.2 are unremarkable. A number of technical proceedures can be inbuilt into the operation of such assays in order to ensure confidence in the recorded scores. Typically all determinations are made at least in triplicate and the mean score may be computed. Where a computed SI=>2.0 individual scores of the triplicate can be examined for evidence of outlying data. Test peptides are contacted with cells in at least two different concentrations and the concentrations would typically span a minimum two-fold concentration difference. Such a concentration range provides an off-set to the kinetic dimension to the assay and is especially important where a single time point determination, for example at plus day 7, is being conducted. In some assays multiple time course determinations may be conducted but in any event these too would be made using peptide immunogen provided at a minimum of two different concentrations. Similarly the inclusion of control peptides for which there is expectation that the majority of PBMC donor samples will be responsive may be included in each assay plate. The influenza haemagglutinin peptide 307-309, sequence PKYVKQNTLKLA; and the Chlamydia HSP 60 peptide sequence KVVDQIKKISKPVQH are particularly suitable control peptides although many other examples may be exploited. Assays should preferably also use a potent whole protein antigen such as hemocyanin from Keyhole Limpet to which all PBMC samples would be expected to exhibit an SI significantly greater than 2.0

It is particularly desired to provide an epitope map of SEB where the map has relevance to a wide spectrum of possible MHC allotypes. It is desired that the map is sufficiently representative to allow the design or selection of a modified protein for which the ability of the protein to evoke a T-cell driven immune response is eliminated or at least ameliorated for the majority of patients to whom the protein is likely to be administered. Accordingly in the practice of the screening process, PBMC derived T-cells from naïve donors is collected from a pool of donors of sufficient immunological diversity to provide a sample of at least greater than 90% of the MHC class II repertoire (HLA-DR) extant in the human population. Where a naïve T-cell response is to be detected to a given synthetic peptide, the peptide in practice is contacted with PBMC preparations derived from multiple donors in isolation, the numbers of donors (or “donor pool” size), is for practical purposes not likely to be less than 20 unrelated individuals and all samples in the donor pool maybe pre-selected according to their MHC class II haplotype.

The term “naïve donor” in the context of the present invention means that the T-cells obtained from the individual who has not been in receipt of any therapeutic sources of SEB, however it is recognised that many individuals in the population may have previously been exposed to environmental sources of exogenous SEB and SEB like proteins. In such individuals there is a likelihood of a recall type response characterised in the context of the present assay by particularly large SI scores. This was indeed found in some individuals where in one instance a particular peptide gave an SI score of 8.1.

The present invention herein discloses a method for T-cell epitope mapping exploiting immunologically naïve T-cells. The T-cells are provided from a peripheral blood sample from a multiplicity of different healthy donors but who have not been in receipt of the protein therapeutically. The assay is conducted using PBMC cultured in vitro using procedures common in the art and involves contacting the PBMC with synthetic peptide species representative of the protein of interest, and following a suitable period of incubation, measurement of peptide induced T cell activation such as cellular proliferation. Measurement is by any suitable means and may for example be conducted using ³H-thymidine incorporation whereby the accumulation of ³H into cellular material is readily measured using laboratory instruments. The degree of cellular proliferation for each combination of PBMC sample and synthetic peptide is examined relative to that seen in non peptide treated PBMC sample. Reference may also be made to the proliferative response seen following treatment with a peptide or peptides for which there is an expected proliferative effect. In this regard it is considered particularly advantageous to use peptide with known broad MHC restriction and especially peptide epitopes with MHC restriction to the DP or DQ isotypes.

To facilitate assembly of an epitope map for SEB, a set of synthetic peptides was produced. Each of the peptides was 15 amino acid residues in length and each overlapped the next peptide in the series by 12 amino acid residues; i.e. each successive peptide in the series incrementally added a further 3 amino acids to the analysis. In this way any given adjacent pair of peptides mapped 18 amino acids of contiguous sequence. For SEB a total of 77 peptides were required to enable a scan of the entire mature protein. However owing to sequence length of the fall protein, to ensure a useful scan of the C-terminus, the final 2 peptides used were a 14 mer and an 11 mer. A particularly effective method for defining a T-cell map for SEB using naïve T-cell assays is provided in the EXAMPLE 1.

The present studies have uncovered 5 peptide sequences able to evoke a significant proliferative response in 2 or more individual donor samples. These peptides are listed in TABLE 1 and are an embodiment of the invention.

Each of the peptides identified in TABLE 1 are suggested to be able to bind MHC class II and engage at least one cognate TCR with sufficient affinity to evoke a proliferative burst detectable in the assay system. These criteria have been achieved using PBMC derived from two or in some cases three unrelated PBMC samples. These peptides are considered to encompass the major epitope regions of the molecule and cluster to three zones in the SEB sequence termed herein epitope regions R1, R2 and R3, or R1a,b,c, R2a and R3a, respectively, which are substrings of the respective strings R1, R2 and R3.

TABLE 1

SEB peptide sequences able to stimulate ex-vivo
human T-cells from 2 or more donor samples

| Peptide | Residue | | Epitope |
|---|---|---|---|
| ID # | #* | Peptide Sequence | Region |
| P6 | 16 | KFTGLMENMKVLYDD | R1a |
| P7 | 19 | GLMENMKVLYDDNHV | R1b |
| P8 | 22 | ENMKVLYDDNHVSAI | R1c |
| P18 | 52 | SIKDTKLGNYDNVRV | R2a |
| P27 | 79 | DKYVDVFGANYYYQC | R3a |

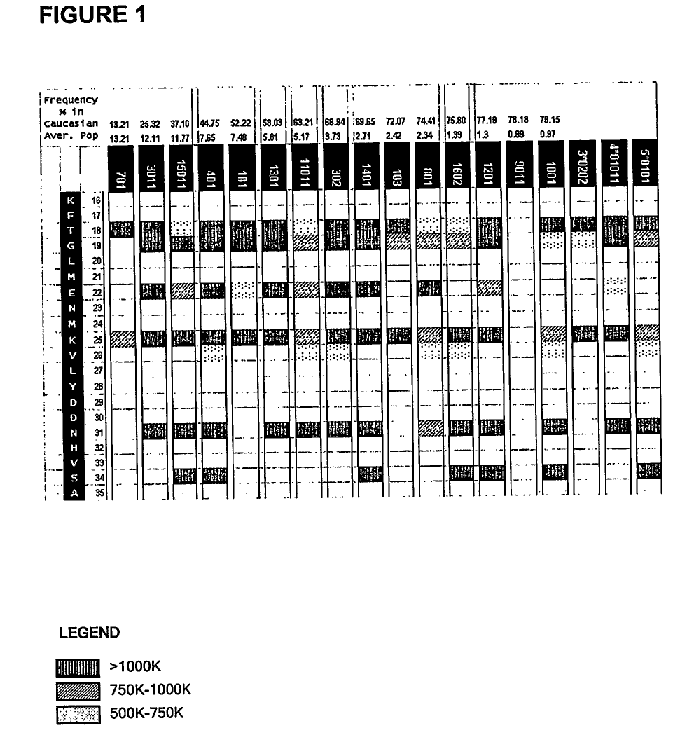

Epitope region R1a is encompassed by peptides P6, P7 and P8 comprising the sequence KFTGLMENMKVLYDDNHVSAI. Note that for the R1a epitope, peptides P6 and P8 are reactive each with two donors samples whereas the intervening peptide P7 is reactive with only one of the donors. In this instance the P7 reaction gave a particularly high SI score (8.1) and reactive sample is also reactive with P6 and P8. Owing to the phasing of each successive peptide in the sequence, it is possible that the same core nonamer sequence could be shared (i.e is common) between either 2 or 3 adjacent peptides. The exact phasing is dependent on proximity to the N-terminus and tied to the length of the peptides and number of “new” residues scanned by each successive increment of the sequence. In the case of the R1a epitope, a number of overlapping MHC class II ligands could be identified (see FIG. 1).

Epitope region R2 is encompassed by peptide P18 comprising the sequence SIKDTKLGNYDNVRV.

Epitope region R3 is encompassed by peptide P27 comprising the sequence DKYVDVFGANYYYQC.

It is understood that further peptide sequences within the SEB sequence could also function as T-cell epitopes, and such sequences may be detected as MHC ligands using physical binding assays in vitro or using virtual means, for example using computational techniques. Additionally, biological assays as provided herein may detect further reacting peptides in particular donor samples, such samples may for example be from individuals recently exposed in the environment to SEB or any other toxin or non-toxin protein containing identical or at least closely homologous peptide sequences to that of SEB. Notwithstanding, it is considered that the disclosed sequences R1a, R2a, R3a herein, represent the critical information required for the construction of modified SEB molecules in which one or more of these epitopes is compromised.

Under the scheme of the present, the epitopes are compromised by mutation to result in sequences no longer able to function as T-cell epitopes. It is possible to use recombinant DNA methods to achieve directed mutagenesis of the target sequences and many such techniques are available and well known in the art.

It is the objective of this invention to modify the amino acid sequences of at least one or more of the above listed peptides from TABLE 1. There are herein disclosed suitable modifications which achieve the objective of reducing or eliminating the capabilities of the subject peptide sequence to function as a T-cell epitope at the level of being a ligand for one or more MHC class II allotypes. One such suitable set of modifications is provided by Formula I.

According to this second embodiment, suitable modifications to the protein may include amino acid substitution of particular residues or combinations of residues. For the elimination of T-cell epitopes, amino acid substitutions are preferably made at appropriate points within the peptide sequence predicted to achieve substantial reduction or elimination of the activity of the T-cell epitope. In practice an appropriate point will preferably equate to an amino acid residue binding within one of the pockets provided within the MHC class II binding groove. It is most preferred to alter binding within the first pocket of the cleft at the so-called “P1” or “P1 anchor” position of the peptide. The quality of binding interaction between the P1 anchor residue of the peptide and the first pocket of the MHC class II binding groove is recognised as being a major determinant of overall binding affinity for the whole peptide. An appropriate substitution at this position of the peptide will be for a residue less readily accommodated within the pocket, for example, substitution to a more hydrophilic residue. Amino acid residues in the peptide at positions equating to binding within other pocket regions within the MHC binding cleft are also considered and fall under the scope of the present.

It is understood that single amino acid substitutions within a given potential T-cell epitope are the most preferred route by which the epitope may be eliminated. Combinations of substitution within a single epitope may be contemplated and for example can be particularly appropriate where individually defined epitopes are in overlap with each other. Moreover, amino acid substitutions either singly within a given epitope or in combination within a single epitope may be made at positions not equating to the “pocket residues” with respect to the MHC class II binding groove, but at any point within the peptide sequence. Substitutions may be made with reference to an homologous structure or structural method produced using in silico techniques known in the art and may be based on known structural features of the molecule. The SEB crystal structure model contained in the Protein Data Bank is particularly useful in this regard [PDB ID: 3SEB Papageoriou, A. C. et al (1998) J. Mol. Biol. 277: 61-79]. A change may be contemplated to restore structure or biological activity of the variant molecule. Such compensatory changes and changes may also include deletion or addition of particular amino acid residues from the polypeptide.

A particularly effective means of removing epitopes from protein molecules is the concerted use of the naïve T-cell activation assay scheme as outlined herein together with an in silico tool developed according to the scheme described in co-owned application WO 02/069232 which is also incorporated fully herein by reference.

The software simulates the process of antigen presentation at the level of the peptide MHC class II binding interaction to provide a binding score for any given peptide sequence. Such a score is determined for many of the predominant MHC class II allotypes extant in the population. As this scheme is able to test any peptide sequence, the consequences of amino acid substitutions additions or deletions with respect to the ability of a peptide to interact with a MHC class II binding groove can be predicted. Consequently new sequence compositions can be designed which contain reduced numbers of peptides able to interact with the MHC class II and thereby function as immmunogenic T-cell epitopes. Where the biological assay using any one given donor sample can assess binding to a maximum of 4 DR allotypes, the in silico process can test the same peptide sequence using >40 allotypes simultaneously. In practice this approach is able to direct the design of new sequence variants which are compromised in the their ability to interact with multiple MHC allotypes.

The T-cell assay was able to define three immunogenic regions R1a-R3a within the molecule and the software system according to the scheme of WO 02/069232 was able to identify predicted MHC class II ligands within each of the epitopes. Moreover, the system was further able to identify amino acid substitutions within the epitopes which resulted in significant loss of binding affinity between the peptide sequence and essentially all of the MHC class II allotypes represented in the system.

One example of such a set of modifications is provided by the disruption of the R1a epitope region. The substitution set M21A, M24A and Y28T result in compromise of the major MHC class II ligands within epitope R1a.

Similarly for MHC class II ligands identified within epitope region R2, the substitutions I53A and L58H are exemplary feasible changes.

For epitope region R3, a suitable substitution series comprises one or more of the changes Y81T, V82H, V84A and F85T.

In all of the above instances, alternative mutation sets can be discerned based on the ability of a given peptide to bind within the MHC class II binding groove and structural considerations based on examination of the SEB crystal structure models PDB ID numbers 3SEB and 1GOZ [for 3SEB see Papageoriou, A. C. et al (1998) J. Mol. Biol. 277: 61-79. For 1GOZ see Baker M. D. et al (2002) J. Biol. Chem. 277:2756-2762].

Each of the above substitutions is exemplary of the method and all are preferred compositions under the scheme of the present invention. As will be clear to the person skilled in the art, multiple alternative sets of substitutions could be arrived at which achieve the objective of removing un-desired epitopes. The resulting sequences would however be recognised to be closely homologous with the specific compositions disclosed herein and therefore fall under the scope of the present invention.

The combined approach of using an in silico tool for the identification of MHC class II ligands and design of sequence analogues lacking MHC class II ligands, in concert with epitope mapping and re-testing optionally using biologically based assays of T-cell activation is a particularly effective method and most preferred embodiment of the invention. The general method according to this embodiment comprises the following steps:
i) use of naïve T-cell activation assays and synthetic peptides collectively encompassing the protein sequence of interest to identify epitope regions capable of activating T-cells;
ii) use of a computational scheme simulating the binding of the peptide ligand with one or more MHC allotypes to analyse the epitope regions identified in step (i) and thereby identify MHC class II ligands within the epitope region;
iii) use of a computational scheme simulating the binding of the peptide ligand with one or more MHC allotypes to identify sequence analogues of the MHC ligands encompassed within the epitope region(s) which no longer bind MHC class II or bind with lowered affinity to a lesser number of MHC allotypes and optionally,
iv) use of naïve T-cell activation assays and synthetic peptides encompassing entirely or in collection encompassing the epitope regions identified within the protein of interest and testing the sequence analogues in naïve T-cell activation assay in parallel with the wild-type (parental) sequences;

The term “T-cell epitope” means according to the understanding of this invention an amino acid sequence which is able to bind MHC class II, able to stimulate T-cells and/or also to bind (without necessarily measurably activating) T-cells in complex with MHC class II.

The term “peptide” as used herein and in the appended claims, is a compound that includes two or more amino acids. The amino acids are linked together by a peptide bond (defined herein below). There are 20 different naturally occurring amino acids involved in the biological production of peptides, and any number of them may be linked in any order to form a peptide chain or ring. The naturally occurring amino acids employed in the biological production of peptides all have the L-configuration. Synthetic peptides can be prepared employing conventional synthetic methods, utilizing L-amino acids, D-amino acids, or various combinations of amino acids of the two different configurations. Some peptides contain only a few amino acid units. Short peptides, e.g., having less than ten amino acid units, are sometimes referred to as “oligopeptides”. Other peptides contain a large number of amino acid residues, e.g. up to 100 or more, and are referred to as “polypeptides”. By convention, a “polypeptide” may be considered as any peptide chain containing three or more amino acids, whereas a “oligopeptide” is usually considered as a particular type of “short” polypeptide. Thus, as used herein, it is understood that any reference to a “polypeptide” also includes an oligopeptide. Further, any reference to a “peptide” includes polypeptides, oligopeptides, and proteins. Each different arrangement of amino acids forms different polypeptides or proteins. The number of polypeptides—and hence the number of different proteins—that can be formed is practically unlimited.

The SEB molecules of this invention can be prepared in any of several ways but is most preferably conducted exploiting routine recombinant methods. It is a relatively facile procedure to use the protein sequences and information provided herein to deduce a polynucleotide (DNA) encoding any of the preferred protein sequences. This can be achieved for example using computer software tools such as the DNSstar software suite [DNAstar Inc, Madison, Wis., USA] or similar. Any such DNA sequence with the capability of encoding the preferred polypeptides of the present or significant homologues thereof, should be considered as embodiments of this invention.

As a general scheme, genes encoding any of the SEB protein sequences can be made using gene synthesis and cloned into a suitable expression vector. In turn the expression vector is introduced into a host cell and cells selected and cultured. The preferred molecules are purified from the culture medium and formulated into a preparation for therapeutic administration. Alternatively, a wild-type SEB gene sequence can be obtained for example following a PCR cloning strategy using DNA from S. aureaus and PCR primers and protocols as set out by Horgan and Fraser [Horgan C & Fraser J. D, In Chapter 8 of MHC Volume 1 A Practical Approach, pp 107-121, Eds: Fernandez, N. & Butcher, G. IRL Press, Oxford 1997]. The wild-type toxin gene can be used as a template for mutagenesis and construction of the preferred variant sequences. In this regard it is particularly convenient to use the strategy of “overlap extension PCR” as described by Higuchi et al [Higuchi et al (1988) Nucleic Acids Res. 16: 7351] although other methodologies and systems could be readily applied. The altered coding DNA is then expressed by conventional means in a selected host cell system such as E. coli, from which the desired SEB is recovered and purified. Suitable host cells, purification and assay schemes are well known in the art.

Where constitution of the SEB molecule may be achieved by recombinant DNA techniques, this may include SEB molecules fused with other protein domains for example an antibody variable region domain. Methods for purifying and manipulating recombinant proteins including fusion proteins are well known in the art. Necessary techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); “Current Protocols in Immunology” (J. E. Coligan et al., eds., 1991).

The invention may be applied to any SEB species of molecule with substantially the same primary amino acid sequences as those disclosed herein and would include therefore SEB molecules derived by genetic engineering means or other processes and may contain more or less than 239 amino acid residues.

Streptococcal enterotoxins A, C, C₁, C₂, D, E and F also other related toxins from different microbial sources have in common many of the peptide sequences of the present disclosure and have in common many peptide sequences with substantially the same sequence as those of the disclosed listing. Such protein sequences equally therefore fall under the scope of the present invention.

In as far as this invention relates to modified SEB, compositions containing such modified SEB proteins or fragments of modified SEB proteins and related compositions should be considered within the scope of the invention. A pertinent example in this respect could be development of peptide mediated tolerance induction strategies wherein one or more of the disclosed peptides is administered to a patient with immunotherapeutic intent. Accordingly, synthetic peptides molecules, for example comprising one of more of the sequences listed in TABLE 1 or more preferably sequences comprising all or part of any of the epitope regions R1a, R2a and R3a are considered embodiments of the invention.

In another aspect, the present invention relates to nucleic acids encoding modified SEB entities. In a further aspect the present invention relates to methods for therapeutic treatment of humans using the modified SEB proteins. In this aspect the modified SEB may be produced as a recombinant fusion protein. In this aspect the modified SEB protein may be linked with an antibody molecule or fragment of an antibody molecule. The linkage may be by means of a chemical cross-linker or more preferably, the SEB-antibody may be produced as a recombinant fusion protein. The fusion molecule may contain the modified SEB domain with antibody domain orientated towards the N-terminus of the fusion molecule although the opposite orientation may be contemplated.

Desired antibody specificities for linkage to the modified SEB molecule of the present include those directed towards cancer specific antigens examples of which include the A33 antigen [Heath, J. K. et al (1997) Proc. Natl, Acad. Sci U.S.A. 94: 469-474] and the GA733-1 antigen [U.S. Pat. No. 5,840,854]. The carcinoembryonic antigen may also be contemplated for use and may be targeted by any of numerous antibodies but may include MFE23 [Chester, K. A. et al (1994) Lancet 343: 455], A5B7 [WO92/010159], T84.66 [U.S. Pat. No. 5,081,235] MN-14 [Hansen, H. J. et al (1993) Cancer 71: 3478-3485], COL-1 [U.S. Pat. No. 5,472,693], the 40 kDa glycoprotein antigen as recognised by antibody KS1/4 [Spearman et al (1987) J. Pharmacol. Exp. Therapeutics 241: 695-703], the epidermal growth factor receptor (HER1) or related receptors such as HER2, anti-GD2 antibodies such as antibody 14.18 [U.S. Pat. No. 4,675,287; EP 0 192 657], or antibodies to the prostate specific membrane antigen [U.S. Pat. No. 6,107,090], the IL-2 receptor [U.S. Pat. No. 6,013,256], the Lewis Y determinant, mucin glycoproteins or others may be contemplated.

In all instances where a modified SEB protein is made in fusion with an antibody sequence it is most desired to use antibody sequences in which T cell epitopes or sequences able to bind MHC class II molecules or stimulate T cells or bind to T cells in association with MHC class II molecules have been removed.

The invention will now be illustrated by the experimental examples below. The examples refer to the following figures:

FIG. 1 is a depiction of the MHC class II ligands identified within epitope region R1a. Ligands are identified using the in silico system of EXAMPLE 2. In this case the binding profile of 18 human DR allotypes are displayed as columns. The ligands detected are 13-mers and residue number 1 of each 13-mer is identified by a coloured block. The intensity of the binding interaction (High, Medium or Low) for each peptide with respect to each of the 18 allotypes is indicated according to the key displayed.

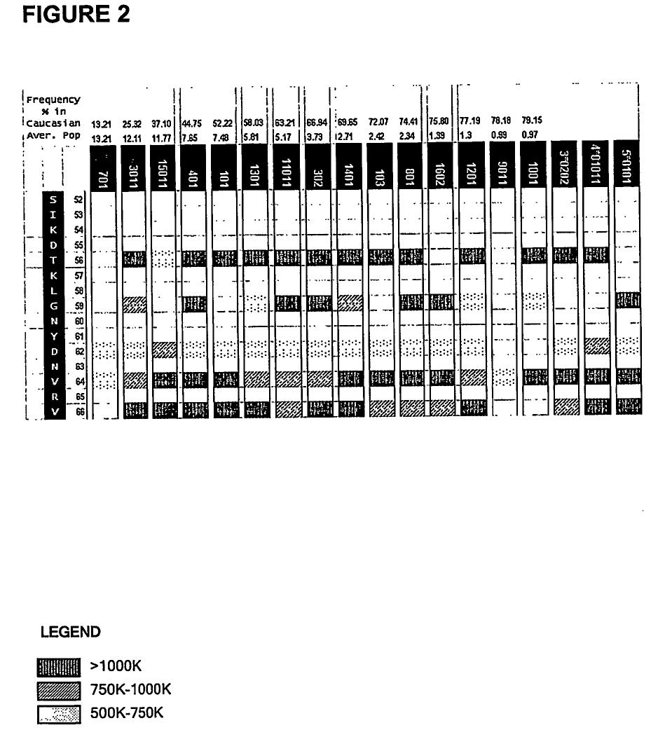

FIG. 2 is a depiction of the MHC class II ligands identified within epitope region R2. Ligands are identified using the in silico system of EXAMPLE 2. In this case the binding profile of 18 human DR allotypes are displayed as columns. The ligands detected are 13-mers and residue number 1 of each 13-mer is identified by a coloured block. The intensity of the binding interaction (High, Medium or Low) for each peptide with respect to each of the 18 allotypes is indicated according to the key displayed.

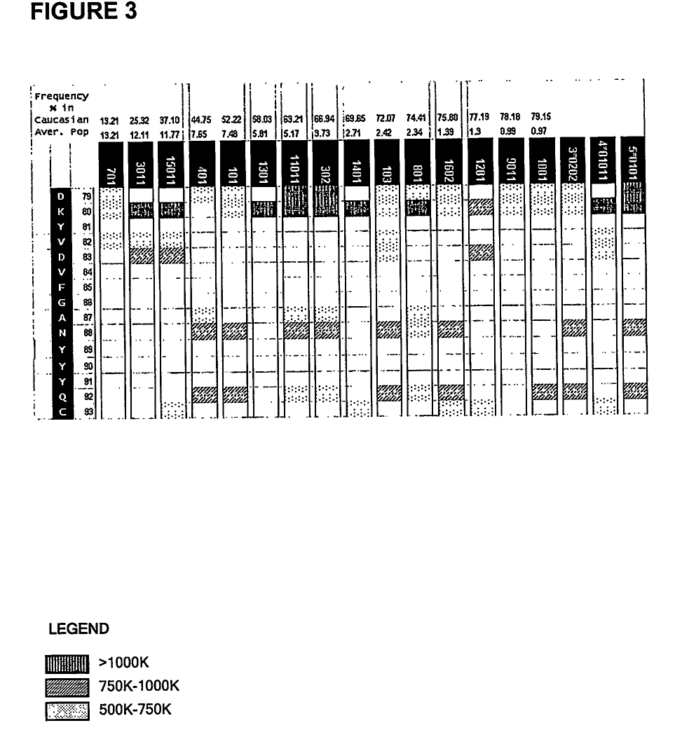

FIG. 3 is a depiction of the MHC class II ligands identified within epitope region R3. Ligands are identified using the in silico system of EXAMPLE 2. In this case the binding profile of 18 human DR allotypes are displayed as columns. The ligands detected are 13-mers and residue number 1 of each 13-mer is identified by a coloured block. The intensity of the binding interaction (High, Medium or Low) for each peptide with respect to each of the 18 allotypes is indicated according to the key displayed.

Formula I (see above) depicts a most preferred SEB structure in which MHC class II ligands are eliminated by substitution within epitope regions R1a, R2a and R3a, and R1a, R1b, R1c, R2a and R3a, respectively.

### EXAMPLE1

The interaction between MHC, peptide and T-cell receptor (TCR) provides the structural basis for the antigen specificity of T-cell recognition. T-cell proliferation assays test the binding of peptides to MHC and the recognition of MHC/peptide complexes by the TCR. In vitro T-cell proliferation assays of the present example, involve the stimulation of peripheral blood mononuclear cells (PBMCs), containing antigen presenting cells (APCs) and T-cells. Stimulation is conducted in vitro using synthetic peptides as antigens. Stimulated T-cell proliferation is measured using ³H-thymidine (³H-Thy) and the presence of incorporated ³H-Thy assessed using scintillation counting of washed fixed cells.

Buffy coats from human blood stored for less than 12 hours were obtained from the National Blood Service (Addenbrooks Hospital, Cambridge, UK). Ficoll-paque was obtained from Amersham Pharmacia Biotech (Amersham, UK). Serum free AIM V media for the culture of primary human lymphocytes and containing L-glutamine, 50 μg/ml streptomycin, 10 μg/ml gentomycin and 0.1% human serum albumin was from Gibco-BRL (Paisley, UK). Synthetic peptides were obtained from Pepscan (The Netherlands) and Babraham Technix (Cambridge, UK).

Erythrocytes and leukocytes were separated from plasma and platelets by gentle centrifugation of buffy coats. The top phase (containing plasma and platelets) was removed and discarded. Erythrocytes and leukocytes were diluted 1:1 in phosphate buffered saline (PBS) before layering onto 15 ml ficoll-paque (Amersham Pharmacia, Amersham UK). Centrifugation was done according to the manufacturers recommended conditions and PBMCs were harvested from the serum+PBS/ficoll paque interface. PBMCs were mixed with PBS (1:1) and collected by centrifugation. The supernatant was removed and discarded and the PBMC pellet re-suspended in 50 ml PBS. Cells were again pelleted by centrifugation and the PBS supernatant discarded. Cells were resuspended using 50 ml AIM V media and at this point counted and viability assessed using trypan blue dye exclusion. Cells were again collected by centrifugation and the supernatant discarded. Cells were re-suspended for cryogenic storage at a density of 3×10⁷ per ml. The storage medium was 90% (v/v) heat inactivated AB human serum (Sigma, Poole, UK) and 10% (v/v) DMSO (Sigma, Poole, UK). Cells were transferred to a regulated freezing container (Sigma) and placed at −70° C. overnight before transferring to liquid N₂ for long term storage. When required for use, cells were thawed rapidly in a water bath at 37° C. before transferring to 10 ml pre-warmed AIM V medium.

PBMC were stimulated with protein and peptide antigens in a 96 well flat bottom plate at a density of 2×10⁵ PBMC per well. PBMC were incubated for 7 days at 37° C. before pulsing with ³H-Thy (Amersham-Phamacia, Amersham, UK). For the present study, synthetic peptides (15mers) that overlapped each successive peptide by 12 amino acids were generated to span the entire sequence of EPO. Peptide identification numbers (ID#) and sequences are given in TABLE 2.

Each peptide was screened individually against PBMC's isolated from 20 naïve donors. Two control peptides that have previously been shown to be immunogenic and a potent non-recall antigen KLH were used in each donor assay. The control antigens used in this study were Flu haemagglutinin 307-319 (sequence: PKYVKQNTLKLAT); Chalamydia HSP 60 peptide (sequence: KVVDQlKKISKPVQH) and Keyhole Limpet hemocyanin. The tissue types for all PBMC samples were assayed using a commercially available reagent system (Iynal, Wirral, UK). Assays were conducted in accordance with the suppliers recommended protocols and standard ancillary reagents and agarose electrophoresis systems.

Peptides were dissolved in DMSO to a final concentration of 10 mM, these stock solutions were then diluted 1/500 in AIM V media (final concentration 20 μM). Peptides were added to a flat bottom 96 well plate to give a final concentration of 2 and 20 μM in a 100 μl. The viability of thawed PBMC's was assessed by trypan blue dye exclusion, cells were then resuspended at a density of 2×10⁶ cells/ml, and 100□1 (2×10⁵ PBMC/weII) was transferred to each well containing peptides. Triplicate well cultures were assayed at each peptide concentration. Plates were incubated for 7 days in a humidified atmosphere of 5% CO₂ at 37° C. Cells were pulsed for 18-21 hours with 1 μCi ³H-Thy/well before harvesting onto filter mats. CPM values were determined using a Wallac microplate beta top plate counter (Perkin Elmer). Results were expressed as stimulation indices, where the stimulation index (SI) is derived by division of the proliferation score (e.g. counts per minute of radioactivity) measured to the test peptide by the score measured in cells not contacted with a test peptide.

TABLE 2

List of SEB synthetic peptides used
for T-cell epitope mapping

| | | |
|---|---|---|
| Peptide | | |
| ID # | SEB; 15 mer peptide sequence | Residue # |

| | | |
|---|---|---|
| P1 | ESQPDPKPDELHKSS | 1 |
| P2 | PDPKPDELHKSSKFT | 4 |
| P3 | KPDELHKSSKFTGLM | 7 |
| P4 | ELHKSSKFTGLMENM | 10 |
| P5 | KSSKFTGLMENMKVL | 13 |
| P6 | KFTGLMENMKVLYDD | 16 |
| P7 | GLMENMKVLYDDNHV | 19 |
| P8 | ENMKVLYDDNHVSAI | 22 |
| P9 | KVLYDDNHVSAINVK | 25 |
| P10 | YDDNHVSAINVKSID | 28 |
| P11 | NHVSAINVKSIDQFL | 31 |
| P12 | SAINVKSIDQFLYFD | 34 |
| P13 | NVKSIDQFLYFDLIY | 37 |
| P14 | SIDQFLYFDLIYSIK | 40 |
| P15 | QFLYFDLIYSIKDTK | 43 |
| P16 | YFDLIYSIKDTKLGN | 46 |
| P17 | LIYSIKDTKLGNYDN | 49 |
| P18 | SIKDTKLGNYDNVRV | 52 |
| P19 | DTKLGNYDNVRVEFK | 55 |
| P20 | LGNYDNVRVEFKNKD | 58 |
| P21 | YDNVRVEFKNKDLAD | 61 |
| P22 | VRVEFKNKDLADKYK | 64 |
| P23 | EFKNKDLADKYKDKY | 67 |
| P24 | NKDLADKYKDKYVDV | 70 |
| P25 | LADKYKDKYVDVFGA | 73 |
| P26 | KYKDKYVDVFGANYY | 76 |
| P27 | DKYVDVFGANYYYQC | 79 |
| P28 | VDVFGANYYYQCYFS | 82 |
| P29 | FGANYYYQCYFSKKT | 85 |
| P30 | NYYYQCYFSKKTNDI | 88 |
| P31 | YQCYFSKKTNDINSH | 91 |
| P32 | YFSKKTNDINSHQTD | 94 |
| P33 | KKTNDINSHQTDKRK | 97 |
| P34 | NDINSHQTDKRKTCM | 100 |
| P35 | NSHQTDKRKTCMYGG | 103 |
| P36 | QTDKRKTCMYGGVTE | 106 |
| P37 | KRKTCMYGGVTEHNG | 109 |
| P38 | TCMYGGVTEHNGNQL | 112 |
| P39 | YGGVTEHNGNQLDKY | 115 |
| P40 | VTEHNGNQLDKYRSI | 118 |
| P41 | HNGNQLDKYRSITVR | 121 |
| P42 | NQLDKYRSITVRVFE | 124 |
| P43 | DKYRSITVRVFEDGK | 127 |
| P44 | RSITVRVFEDGKNLL | 130 |
| P45 | TVRVFEDGKNLLSFD | 133 |
| P46 | VFEDGKNLLSFDVQT | 136 |
| P47 | DGKNLLSFDVQTNKK | 139 |
| P48 | NLLSFDVQTNKKKVT | 142 |
| P49 | SFDVQTNKKKVTAQE | 145 |
| P50 | VQTNKKKVTAQELDY | 148 |
| P51 | NKKKVTAQELDYLTR | 151 |
| P52 | KVTAQELDYLTRHYL | 154 |
| P53 | AQELDYLTRHYLVKN | 157 |
| P54 | LDYLTRHYLVKNKKL | 160 |
| P55 | LTRHYLVKNKKLYEF | 163 |
| P56 | HYLVKNKKLYEFNNS | 166 |
| P57 | VKNKKLYEFNNSPYE | 169 |
| P58 | KKLYEFNNSPYETGY | 172 |
| P59 | YEFNNSPYETGYIKF | 175 |
| P60 | NNSPYETGYIKFIEN | 178 |
| P61 | PYETGYLKFIENENS | 181 |
| P62 | TGYIKFIENENSFWY | 184 |
| P63 | IKFIENENSFWYDMM | 187 |
| P64 | IENENSFWYDMMPAP | 190 |
| P65 | ENSFWYDMMPAPGDK | 193 |
| P66 | FWYDMMPAPGDKFDQ | 196 |
| P67 | DMMPAPGDKFDQSKY | 199 |
| P68 | PAPGDKFDQSKYLMM | 202 |
| P69 | GDKFDQSKYLMMYND | 205 |
| P70 | FDQSKYLMMYNDNKM | 208 |
| P71 | SKYLMMYNDNKMVDS | 211 |
| P72 | LMMYNDNKMVDSKDV | 214 |
| P73 | YNDNKMVDSKDVKIE | 217 |
| P74 | NKMVDSKDVKIEVYL | 220 |
| P75 | VDSKDVKIEVYLTTK | 223 |
| P76 | KDVKIEVYLTTKKK | 226 |
| P77 | KIEVYLTTKKK | 229 |

Mapping T cell epitopes in the SEB sequence using the T cell proliferation assay resulted in the identification of 3 immunogenic regions R1a, R2a and R3a. Peptides able to stimulate a significant response are listed within TABLE 1. The allotypic restriction of responsive donors and the recorded SI to SEB peptides is given in TABLE 3.

TABLE 3

| | | SI per | |
|---|---|---|---|
| Pep- | | respon- | |
| tide | | sive | |
| ID # | Peptide Sequence | sample* | Responsive Allotypes |
| P6 | KFTGLMENMKVLYDD | 3.4, | DRB1*04, DRB4*01; DRB1*07, |
| | | 2.1 | DRB1*11, DRB3 |
| P7 | GLMENMKVLYDDNHV | 8.1 | DRB1*Q4, DRB4*01 |
| P8 | ENMKVLYDDNHVSAI | 4.4 | DRB1*04, DRB4*01; DRBI*07, |
| | | 3.1 | DRB1*09, DRB4*01 |
| P18 | SIKDTKLGNYDNVRV | 2.2 | DRB1*04, DRB4*01; DRB1*07, |
| | | 5.1 | DRB1*09, DRB1*12, DRB1*15, |
| | | 2.0 | DRB3, DRB5 |
| P27 | DKYVDVFGANYYYQC | 2.3 | DRB1*04, DRB4*01; DRB1*07, |
| | | 5.3 | DRB1*09, DRB1*07, |
| | | 2.5 | DRB1*11, DRB3 |

*SI= Stimulation index. The figure given is the mean of triplicate determinations for each responsive donor sample. All peptides were tested at 1uM and 5uM. The SI given relates to the higher of the two determinations.

### EXAMPLE 2

Design of Modified SEB Sequences with Improved Immunogenicity Profiles:

The method of co-owned application WO 02/069232 was used in an analysis of the epitope regions R1a, R2a and R3a The system enables prediction of the particular MHC ligands encompassed within the biologically detected epitope regions and provides a “score” with respect to the ability of a given MHC class II ligand to interact with a particular MHC allotype.

The allotypic restriction pattern for the MHC ligands can be depicted using the allotypic restriction chart displays as provided for each of the epitope regions R1a-R3a in the accompanying FIGS. 1-3.

The analysis was extended to consideration of sequence modifications within each of the epitopes R1a -R3a The sequence variants were tested for continued ability bind MHC class II and their binding scores where these remained. Multiple amino acid substitutions were defined which achieved elimination of MHC class II binding with the majority of MHC allotypes tested. The particular substitutions identified were further tested for their ability to be accommodated within the[SEB crystal structure models PDB ID numbers 3SEB and 1GOZ [for 3SEB see Papageoriou, A. C. et al (1998) J. Mol. Biol. 277: 61-79. For 1GOZ see Baker M. D. et al (2002) J. Biol. Chem. 277:2756-2762]. Designed mutations on the selected residues of the wild type sequence were checked for steric clashes, hydrogen bonding formation, hydrophobic interactions and its general accommodation in the structure. Substitutions that gave rise to steric clashes were dismissed. Substitutions that were accommodated when the side chain was adopting a similar configuration (rotamer) to the original residue were considered acceptable. If more than one substitution fulfilled these criteria, residues that potentially form hydrogen bonds with neighboring side chains or backbone atoms, and/or form favourable hydrophobic contacts or other associations were preferred. The above procedure was performed interactively using Swiss Prot Deep View v3.7 [Guex, N. and Peitsch, M. C. (1997) Electrophoresis 18: 2714-2723]. This process resulted in a preferred substitution set for each of the epitope regions R1-R3, preferably R1a -R3a. The substitution sets were compiled to produce the structure depicted in Formula I. All substitutions were confirmed to result in removal of the MHC class II ligands within each of the epitope regions R1-R3, preferably R1a-R3a. A SEB structure containing the most preferred set of substitutions according to the above scheme is depicted as Formula I.

1. A modified molecule having the biological activity of staphylococcal enterotoxin B (SEB) and being substantially non-immunogenic or less immunogenic than any non-modified molecule having the same biological activity in an individual when used in vivo, wherein (i) the said loss of immunogenicity is achieved by removing one or more T-cell epitopes derived from the originally non-modified molecule and said T-cell epitopes are MHC class II ligands or peptide sequences which show the ability to stimulate or bind T-cells via presentation on class II,
(ii) said modified molecule, when tested as a whole protein in a biological human T-cell proliferation assay, exhibits a stimulation index (SI) smaller than the parental non-modified molecule and smaller than 2.0, and
(iii) said T-cell epitopes to be removed are located on one or more strings termed R1 to R3 of contiguous residues of the originally non-modified SEB molecule, the strings are selected from:
(SEQ ID NO: 2)
R1: KFTGLMENMKVLYDDNHVSAI;
(SEQ ID NO: 3)
R2: QFLYFDLIYSLKDTKLGNYDNVRV;
(SEQ ID NO: 4)
R3: NKDLADKYKDKYVDVFGANYYYQCYFSKKTNDI.

2-16. (canceled)

17. An isolated polypeptide having the biological activity of native staphylococcal enterotoxin B and being less immunogenic to a human than native staphylococcal enterotoxin B, the polypeptide comprising the amino acid residue sequence of SEQ ID NO: 1 and including at least one amino acid residue substitution in at least one epitope region of SEQ ID NO: 1 selected from the group consisting of
(R1) amino acid residues 16-36 of SEQ ID NO: 1,
(R2) amino acid residues 43-66 of SEQ ID NO: 1, and
(R3) amino acid residues 70-102 of SEQ ID NO: 1.

18. The isolated polypeptide of claim 17 wherein the at least one amino acid residue substitution in epitope region (R1) is selected from the group consisting of: Met21Ala, Met21Gly, Met21Pro, Met24Ala, Met24Gly, Met24Pro, Tyr28Thr, Tyr28Ala, Tyr28Asp, Tyr28Glu, Tyr28Gly, Tyr28His, Tyr28Lys, Tyr28Asn, Tyr28Asn, Tyr28Pro, Tyr28Gln, Tyr28Arg, and Tyr28Ser.

19. The isolated polypeptide of claim 17 wherein the at least one amino acid residue substitution in epitope region (R2) is selected from the group consisting of: II353Ala and Leu58His.

20. The isolated polypeptide of claim 17 wherein the at least one amino acid residue substitution in epitope region (R3) is selected from the group consisting of: Tyr81Thr, Tyr81Ala, Tyr81Asp, Tyr81Glu, Tyr818Gly, Tyr81His, Tyr81Lys, Tyr81Asn, Tyr81Asn, Tyr81Pro, Tyr81Gln, Tyr81Arg, Tyr81Ser, Val82His, Val84Ala, Val84Pro, Val84Gly, Phe85Thr, and Phe85His.

21. An isolated polypeptide having the biological activity of native staphylococcal enterotoxin B and being less immunogenic to a human than native staphylococcal enterotoxin B, the polypeptide comprising the amino acid residue sequence of SEQ ID NO: 1 and including at least one amino acid residue substitution in at least one epitope region of SEQ ID NO: 1 selected from the group consisting of:
(R1a) amino acid residues 16-30 of SEQ ID NO: 1,
(R1b) amino acid residues 19-33 of SEQ ID NO: 1,
(R1c) amino acid residues 22-36 of SEQ ID NO: 1,
(R2a) amino acid residues 52-66 of SEQ ID NO: 1, and
(R3a) amino acid residues 79-93 of SEQ ID NO: 1.

22. The isolated polypeptide of claim 21 wherein the at least one amino acid residue substitution in epitope region (R1a) is selected from the group consisting of: Met21Ala, Met21Gly, Met21Pro, Met24Ala, Met24Gly, Met24Pro, Tyr28Thr, Tyr28Ala, Tyr28Asp, Tyr28Glu, Tyr28Gly, Tyr28His, Tyr28Lys, Tyr28Asn, Tyr28Asn, Tyr28Pro, Tyr28Gln, Tyr28Arg, and Tyr28Ser.

23. The isolated polypeptide of claim 21 wherein the at least one amino acid residue substitution in epitope region (R1b) is selected from the group consisting of: Met21Ala, Met21Gly, Met21Pro, Met24Ala, Met24Gly, Met24Pro, Tyr28Thr, Tyr28Ala, Tyr28Asp, Tyr28Glu, Tyr28Gly, Tyr28His, Tyr28Lys, Tyr28Asn, Tyr28Asn, Tyr28Pro, Tyr28Gln, Tyr28Arg, and Tyr28Ser.

24. The isolated polypeptide of claim 21 wherein the at least one amino acid residue substitution in epitope region (R1c) is selected from the group consisting of: Met24Ala, Met24Gly, Met24Pro, Tyr28Thr, Tyr28Ala, Tyr28Asp, Tyr28Glu, Tyr28Gly, Tyr28His, Tyr28Lys, Tyr28Asn, Tyr28Asn, Tyr28Pro, Tyr28Gln, Tyr28Arg, and Tyr28Ser.

25. The isolated polypeptide of claim 21 wherein the at least one amino acid residue substitution in epitope region (R2a) is selected from the group consisting of: Il353Ala and Leu58His.

25. The isolated polypeptide of claim 21 wherein the at least one amino acid residue substitution in epitope region (R3a) is selected from the group consisting of: Tyr81Thr, Tyr81Ala, Tyr81Asp, Tyr81Glu, Tyr818Gly, Tyr81His, Tyr81Lys, Tyr81Asn, Tyr81Asn, Tyr81Pro, Tyr81Gln, Tyr81Arg, Tyr81Ser, Val82His, Val84Ala, Val84Pro, Val84Gly, Phe85Thr, and Phe85His.

26. An isolated polypeptide comprising the amino acid residue sequence of SEQ ID NO: 1 and including at least one amino acid residue substitution in SEQ ID NO: 1 selected from the group consisting of: Met21Ala, Met21Gly, Met21Pro, Met24Ala, Met24Gly, Met24Pro, Tyr28Thr, Tyr28Ala, Tyr28Asp, Tyr28Glu, Tyr28Gly, Tyr28His, Tyr28Lys, Tyr28Asn, Tyr28Asn, Tyr28Pro, Tyr28Gln, Tyr28Arg, Tyr28Ser, Il353Ala, Leu58His, Tyr81Thr, Tyr81Ala, Tyr81Asp, Tyr81Glu, Tyr818Gly, Tyr81His, Tyr81Lys, Tyr81Asn, Tyr81Asn, Tyr81Pro, Tyr81Gln, Tyr81Arg, Tyr81Ser, Val82His, Val84Ala, Val84Pro, Val84Gly, Phe85Thr, and Phe85His.

27. An isolated nucleic acid that encodes a polypeptide of claim 17.

28. An isolated nucleic acid that encodes a polypeptide of claim 21.

29. An isolated nucleic acid that encodes a polypeptide of claim 26.

30. A pharmaceutical composition comprising a polypeptide of claim 17 in a pharmaceutically acceptable carrier therefor.

31. A pharmaceutical composition comprising a polypeptide of claim 21 in a pharmaceutically acceptable carrier therefor.

32. A pharmaceutical composition comprising a polypeptide of claim 26 in a pharmaceutically acceptable carrier therefor.

33. The isolated polypeptide of claim 17 wherein the polypeptide exhibits a stimulation index of less than 2 when tested in a biological human T-cell proliferation assay.

34. The isolated polypeptide of claim 17 wherein the polypeptide exhibits a stimulation index of less than 1.8 when tested in a biological human T-cell proliferation assay.

35. The isolated polypeptide of claim 29 wherein the polypeptide exhibits a stimulation index of less than 2 when tested in a biological human T-cell proliferation assay.

36. The isolated polypeptide of claim 29 wherein the polypeptide exhibits a stimulation index of less than 1.8 when tested in a biological human T-cell proliferation assay.


SEQUENCE LISTING (84 sequences)

SEQ ID NO: 1 - protein, 239 aa, Staphylococcus aureus
ESQPDPKPDELHKSSKFTGLMENMKVLYDDNHVSAINVKSIDQFLYFDLIYSIKDTKLGNYDNVRVEFKNKDLADKYKDKYVDVFGANYYYQCYFSKKTNDINSHQTDKRKTCMYGGVTEHNGNQLDKYRSITVRVFEDGKNLLSFDVQTNKKKVTAQELDYLTRHYLVKNKKLYEFNNSPYETGYIKFIENENSFWYDMMPAPGDKFDQSKYLMMYNDNKMVDSKDVKIEVYLTTKKK

SEQ ID NO: 2 - protein, 21 aa, Staphylococcus aureus
KFTGLMENMKVLYDDNHVSAI

SEQ ID NO: 3 - protein, 24 aa, Staphylococcus aureus
QFLYFDLIYSIKDTKLGNYDNVRV

SEQ ID NO: 4 - protein, 33 aa, Staphylococcus aureus
NKDLADKYKDKYVDVFGANYYYQCYFSKKTNDI

SEQ ID NO: 5 - protein, 239 aa, Artificial Sequence
  Modified enterotoxin protein
ESQPDPKPDELHKSSKFTGLXENXKVLXDDNHVSAINVKSIDQFLYFDLIYSXKDTKXGNYDNVRVEFKNKDLADKYKDKXXDXXGANYYYQCYFSKKTNDINSHQTDKRKTCMYGGVTEHNGNQLDKYRSITVRVFEDGKNLLSFDVQTNKKKVTAQELDYLTRHYLVKNKKLYEFNNSPYETGYIKFIENENSFWYDMMPAPGDKFDQSKYLMMYNDNKMVDSKDVKIEVYLTTKKK

SEQ ID NO: 6 - protein, 12 aa, Staphylococcus aureus
PKYVKQNTLKLA

SEQ ID NO: 7 - protein, 15 aa, Staphylococcus aureus
KVVDQIKKISKPVQH

SEQ ID NO: 8 - protein, 15 aa, Staphylococcus aureus
ESQPDPKPDELHKSS

SEQ ID NO: 9 - protein, 15 aa, Staphylococcus aureus
PDPKPDELHKSSKFT

SEQ ID NO: 10 - protein, 15 aa, Staphylococcus aureus
KPDELHKSSKFTGLM

SEQ ID NO: 11 - protein, 15 aa, Staphylococcus aureus
ELHKSSKFTGLMENM

SEQ ID NO: 12 - protein, 15 aa, Staphylococcus aureus
KSSKFTGLMENMKVL

SEQ ID NO: 13 - protein, 15 aa, Staphylococcus aureus
KFTGLMENMKVLYDD

SEQ ID NO: 14 - protein, 15 aa, Staphylococcus aureus
GLMENMKVLYDDNHV

SEQ ID NO: 15 - protein, 15 aa, Staphylococcus aureus
ENMKVLYDDNHVSAI

SEQ ID NO: 16 - protein, 15 aa, Staphylococcus aureus
KVLYDDNHVSAINVK

SEQ ID NO: 17 - protein, 15 aa, Staphylococcus aureus
YDDNHVSAINVKSID

SEQ ID NO: 18 - protein, 15 aa, Staphylococcus aureus
NHVSAINVKSIDQFL

SEQ ID NO: 19 - protein, 15 aa, Staphylococcus aureus
SAINVKSIDQFLYFD

SEQ ID NO: 20 - protein, 15 aa, Staphylococcus aureus
NVKSIDQFLYFDLIY

SEQ ID NO: 21 - protein, 15 aa, Staphylococcus aureus
SIDQFLYFDLIYSIK

SEQ ID NO: 22 - protein, 15 aa, Staphylococcus aureus
QFLYFDLIYSIKDTK

SEQ ID NO: 23 - protein, 15 aa, Staphylococcus aureus
YFDLIYSIKDTKLGN

SEQ ID NO: 24 - protein, 15 aa, Staphylococcus aureus
LIYSIKDTKLGNYDN

SEQ ID NO: 25 - protein, 15 aa, Staphylococcus aureus
SIKDTKLGNYDNVRV

SEQ ID NO: 26 - protein, 15 aa, Staphylococcus aureus
DTKLGNYDNVRVEFK

SEQ ID NO: 27 - protein, 15 aa, Staphylococcus aureus
LGNYDNVRVEFKNKD

SEQ ID NO: 28 - protein, 15 aa, Staphylococcus aureus
YDNVRVEFKNKDLAD

SEQ ID NO: 29 - protein, 15 aa, Staphylococcus aureus
VRVEFKNKDLADKYK

SEQ ID NO: 30 - protein, 15 aa, Staphylococcus aureus
EFKNKDLADKYKDKY

SEQ ID NO: 31 - protein, 15 aa, Staphylococcus aureus
NKDLADKYKDKYVDV

SEQ ID NO: 32 - protein, 15 aa, Staphylococcus aureus
LADKYKDKYVDVFGA

SEQ ID NO: 33 - protein, 15 aa, Staphylococcus aureus
KYKDKYVDVFGANYY

SEQ ID NO: 34 - protein, 15 aa, Staphylococcus aureus
DKYVDVFGANYYYQC

SEQ ID NO: 35 - protein, 15 aa, Staphylococcus aureus
VDVFGANYYYQCYFS

SEQ ID NO: 36 - protein, 15 aa, Staphylococcus aureus
FGANYYYQCYFSKKT

SEQ ID NO: 37 - protein, 15 aa, Staphylococcus aureus
NYYYQCYFSKKTNDI

SEQ ID NO: 38 - protein, 15 aa, Staphylococcus aureus
YQCYFSKKTNDINSH

SEQ ID NO: 39 - protein, 15 aa, Staphylococcus aureus
YFSKKTNDINSHQTD

SEQ ID NO: 40 - protein, 15 aa, Staphylococcus aureus
KKTNDINSHQTDKRK

SEQ ID NO: 41 - protein, 15 aa, Staphylococcus aureus
NDINSHQTDKRKTCM

SEQ ID NO: 42 - protein, 15 aa, Staphylococcus aureus
NSHQTDKRKTCMYGG

SEQ ID NO: 43 - protein, 15 aa, Staphylococcus aureus
QTDKRKTCMYGGVTE

SEQ ID NO: 44 - protein, 15 aa, Staphylococcus aureus
KRKTCMYGGVTEHNG

SEQ ID NO: 45 - protein, 15 aa, Staphylococcus aureus
TCMYGGVTEHNGNQL

SEQ ID NO: 46 - protein, 15 aa, Staphylococcus aureus
YGGVTEHNGNQLDKY

SEQ ID NO: 47 - protein, 15 aa, Staphylococcus aureus
VTEHNGNQLDKYRSI

SEQ ID NO: 48 - protein, 15 aa, Staphylococcus aureus
HNGNQLDKYRSITVR

SEQ ID NO: 49 - protein, 15 aa, Staphylococcus aureus
NQLDKYRSITVRVFE

SEQ ID NO: 50 - protein, 15 aa, Staphylococcus aureus
DKYRSITVRVFEDGK

SEQ ID NO: 51 - protein, 15 aa, Staphylococcus aureus
RSITVRVFEDGKNLL

SEQ ID NO: 52 - protein, 15 aa, Staphylococcus aureus
TVRVFEDGKNLLSFD

SEQ ID NO: 53 - protein, 15 aa, Staphylococcus aureus
VFEDGKNLLSFDVQT

SEQ ID NO: 54 - protein, 15 aa, Staphylococcus aureus
DGKNLLSFDVQTNKK

SEQ ID NO: 55 - protein, 15 aa, Staphylococcus aureus
NLLSFDVQTNKKKVT

SEQ ID NO: 56 - protein, 15 aa, Staphylococcus aureus
SFDVQTNKKKVTAQE

SEQ ID NO: 57 - protein, 15 aa, Staphylococcus aureus
VQTNKKKVTAQELDY

SEQ ID NO: 58 - protein, 15 aa, Staphylococcus aureus
NKKKVTAQELDYLTR

SEQ ID NO: 59 - protein, 15 aa, Staphylococcus aureus
KVTAQELDYLTRHYL

SEQ ID NO: 60 - protein, 15 aa, Staphylococcus aureus
AQELDYLTRHYLVKN

SEQ ID NO: 61 - protein, 15 aa, Staphylococcus aureus
LDYLTRHYLVKNKKL

SEQ ID NO: 62 - protein, 15 aa, Staphylococcus aureus
LTRHYLVKNKKLYEF

SEQ ID NO: 63 - protein, 15 aa, Staphylococcus aureus
HYLVKNKKLYEFNNS

SEQ ID NO: 64 - protein, 15 aa, Staphylococcus aureus
VKNKKLYEFNNSPYE

SEQ ID NO: 65 - protein, 15 aa, Staphylococcus aureus
KKLYEFNNSPYETGY

SEQ ID NO: 66 - protein, 15 aa, Staphylococcus aureus
YEFNNSPYETGYIKF

SEQ ID NO: 67 - protein, 15 aa, Staphylococcus aureus
NNSPYETGYIKFIEN

SEQ ID NO: 68 - protein, 15 aa, Staphylococcus aureus
PYETGYIKFIENENS

SEQ ID NO: 69 - protein, 15 aa, Staphylococcus aureus
TGYIKFIENENSFWY

SEQ ID NO: 70 - protein, 15 aa, Staphylococcus aureus
IKFIENENSFWYDMM

SEQ ID NO: 71 - protein, 15 aa, Staphylococcus aureus
IENENSFWYDMMPAP

SEQ ID NO: 72 - protein, 15 aa, Staphylococcus aureus
ENSFWYDMMPAPGDK

SEQ ID NO: 73 - protein, 15 aa, Staphylococcus aureus
FWYDMMPAPGDKFDQ

SEQ ID NO: 74 - protein, 15 aa, Staphylococcus aureus
DMMPAPGDKFDQSKY

SEQ ID NO: 75 - protein, 15 aa, Staphylococcus aureus
PAPGDKFDQSKYLMM

SEQ ID NO: 76 - protein, 15 aa, Staphylococcus aureus
GDKFDQSKYLMMYND

SEQ ID NO: 77 - protein, 15 aa, Staphylococcus aureus
FDQSKYLMMYNDNKM

SEQ ID NO: 78 - protein, 15 aa, Staphylococcus aureus
SKYLMMYNDNKMVDS

SEQ ID NO: 79 - protein, 15 aa, Staphylococcus aureus
LMMYNDNKMVDSKDV

SEQ ID NO: 80 - protein, 15 aa, Staphylococcus aureus
YNDNKMVDSKDVKIE

SEQ ID NO: 81 - protein, 15 aa, Staphylococcus aureus
NKMVDSKDVKIEVYL

SEQ ID NO: 82 - protein, 15 aa, Staphylococcus aureus
VDSKDVKIEVYLTTK

SEQ ID NO: 83 - protein, 14 aa, Staphylococcus aureus
KDVKIEVYLTTKKK

SEQ ID NO: 84 - protein, 11 aa, Staphylococcus aureus
KIEVYLTTKKK